(19) United States
(12) Patent Application Publication
(10) Pub. No.: US 2020/0323952 A1
(43) Pub. Date: Oct. 15, 2020

(54) COMPOSITIONS AND METHODS OF TREATING CANCER

(71) Applicants: Dana-Farber Cancer Institute, Inc., Boston, MA (US); Beth Israel Deaconess Medical Center, Boston, MA (US)

(72) Inventors: David Avigan, Sharon, MA (US); Jacalyn Rosenblatt, Newton, MA (US); Donald Kufe, Wellesley, MA (US)

(21) Appl. No.: 15/776,369

(22) PCT Filed: Nov. 21, 2016
(86) PCT No.: PCT/US16/63109

§ 371 Date: May 15, 2018

Related U.S. Application Data
(60) Provisional application No. 62/257,945, filed on Nov. 20, 2015.

(51) Int. Cl. A61K 38/17; A61P 35/00; A61K 45/06; A61K 31/454; A61K 31/4035

(52) U.S. Cl. CPC A61K 38/1709; A61P 35/00; A61K 31/4035; A61K 31/454; A61K 45/06

(57) ABSTRACT

The present invention provides compositions and methods for treating cancer.

### RELATED APPLICATIONS

This application claims priority to, and the benefit of, U.S. Provisional Application No. 62/257,945, filed Nov. 20, 2015, the contents of which are incorporated herein by reference in their entirety.

### GOVERNMENT INTEREST

This invention was made with government support under grant numbers CA100707 and CA078378 awarded by The National Institutes of Health. The government has certain rights in the invention.

### FIELD OF THE INVENTION

The present invention relates generally to cellular immunology and more particularly to and methods for treating cancer by inhibiting PD-L1 via MUC-1 inhibition.

### BACKGROUND OF THE INVENTION

In health, the PD-L1/PD-1 pathway provides a critical negative costimulatory signal in the complex interaction between antigen presenting and effector cells acting as a counter-regulatory influence to prevent over-activation of T cells and immune mediated damage. Cancer cells markedly upregulate PD-L1 expression resulting in an immunosuppressive milieu in the tumor microenvironment. PD-L1 ligation of PD-1 on T cells induces an exhausted phenotype characterized by loss of T cell activation and expansion and blunting of effector mediated targeted killing of tumor cells. Recent clinical studies have shown that antibody blockade of PD-1 or PD-L1 results in dramatic and durable disease response in patients with advanced solid tumors and hematological malignancies that were no longer responsive to cytotoxic chemotherapy. Despite its emergence as a critical target in cancer therapeutics, little is known about the oncogenic modulation of PD-L1 expression.

### SUMMARY OF THE INVENTION

The invention features methods of treating a tumor in a patient by administering to the patient a composition containing a MUC1 inhibitor in an amount sufficient to decrease tumor PD-L1 expression. Optionally, the patient is further administered a checkpoint inhibitor. In various aspects the patient has received a population of autologous dendritic cell/tumor cell fusions (DC/tumor fusions). The MUC1 inhibitor is for example GO-203.

The tumor is a solid tumor or a hematologic tumor. For example, the solid tumor is a lung tumor, a breast tumor, or a renal tumor. The hematologic tumor is for example, acute myeloid leukemia (AML) or multiple myeloma (MM).

Exemplary checkpoint inhibitors include an A2AR, B7-H3/CD276, B7-H4/VTCN1, BTLA, CD27, CD28, CD40, CD122, CD137, CTLA-4, GITR, ICOS, IDO, KIR, LAG3, OX40, PD1, PD-L1, PD-L2, TIM-3, or VISTA inhibitor. Preferably, the checkpoint inhibitor is an A2AR, B7-H3/CD276, B7-H4/VTCN1, BTLA, CD27, CD28, CD40, CD122, CD137, CTLA-4, GITR, ICOS, IDO, KIR, LAG3, OX40, PD1, PD-L1, PD-L2, TIM-3, or VISTA antibody.

In various aspects the method further includes administering to the patient an agent that targets regulatory T cells, an immunomodulatory agent or both. The immunomodulatory agent is lenalidomide, pomalinomide, or apremilast.

In other aspects the patient is administering a TLR agonist, CPG ODN, polyIC, or tetanus toxoid.

Unless otherwise defined, all technical and scientific terms used herein have the same meaning as commonly understood by one of ordinary skill in the art to which this invention pertains. Although methods and materials similar or equivalent to those described herein can be used in the practice of the present invention, suitable methods and materials are described below. All publications, patent applications, patents, and other references mentioned herein are expressly incorporated by reference in their entirety. In cases of conflict, the present specification, including definitions, will control. In addition, the materials, methods, and examples described herein are illustrative only and are not intended to be limiting. Other features and advantages of the invention will be apparent from and encompassed by the following detailed description and claims.

### BRIEF DESCRIPTION OF THE DRAWINGS

FIG. 1. MUC1-C regulates PDL-1 expression by controlling miR-200c micro-RNA. a. RPMI-8226 Multiple Myeloma (MM) cancer cells stably expressing a Control shRNA (CshRNA) or a MUC1 shRNA (MUC1shRNA) were analyzed for MUC1 and PDL-1 mRNA levels by qRT-PCR. The results (mean±SD of 3 determinations) are expressed as relative mRNA levels compared to that obtained with cells expressing MUC1shRNA. Lysates from the RPMI/CshRNA and RPMI/MUC1shRNA cells were immunoblotted with the indicated antibodies. b. U266 multiple myeloma cancer cells stably expressing a CshRNA or MUC1shRNA were analyzed for (i) MUC1 and PDL-1 mRNA and (ii) protein. c. RPMI-Multiple Myeloma (MM) cancer cells stably expressing a Control shRNA (CshRNA) or a MUC1 shRNA (MUC1shRNA) were analyzed for miR200-c micro-RNA levels by qRT-PCR and PDL-1 protein levels by FACS (left) and immunoblotted with the indicated antibodies (right). The results (mean±SD of 3 determinations) are expressed as relative mRNA levels compared to that obtained with cells expressing MUC1shRNA. d. U266 multiple myeloma cancer cells stably expressing a CshRNA or MUC1shRNA were analyzed for (i) miR-200c micro-RNA and (ii) PDL-1 protein by FACS and (iii) immunoblotted with indicated antibodies.

FIG. 2. Ectopic expression of miR-200c downregulates PDL-1 expression. a. schema of PDL-1 3′UTR indicating miR-200c binding site. The indicated RPMI (left) and U266 (right) cells were transfected with the empty 3′UTR-renila vector or PDL-1 3′UTR-renila vector. Renila Luciferase activity was measured at 48 h after transfection. The results (mean±SD of 3 determinations) are expressed as the relative luciferase activity compared to that obtained with the cell expressing Control-shRNA (assigned a value of 1). b. The indicated RPMI(left) and U266 (right) cells were immunoblotted with the indicated antibodies.

FIG. 3 MUC1-C regulates PDL-1 expression by controlling miR-200c micro-RNA.

FIG. 4. SNAI-1 (Snail) occupies the miR-200c promoter in a MUC1 dependent manner. a. Soluble chromatin from RPMI cells stably expressing a Control shRNA (CshRNA) or a MUC1 shRNA (MUC1shRNA) were precipitated with anti-SNAI-1 (left) or a control IgG. b. soluble chromatin from U266 cells stably expressing a Control shRNA (CshRNA) or a MUC1 shRNA (MUC1shRNA) were precipitated with anti-SNAI-1 snail (right) or a control IgG. The final DNA samples were amplified by qPCR with pairs of primers for the snail binding site in the miR-200c promoter. The results (mean±SD of 3 determinations) are expressed as the relative fold enrichment compared with that obtained for the MUC1 shRNA (assigned a value of 1). c. A Co-IP with Snail antibody and immunoblotting with MUC1-C in (i) RPMI or (ii) U266 cells as compared to Co-IP with the IgG control.

FIG. 5. MUC1-C silencing increases the cell susceptibility towards non-autologous T-cell.

FIG. 6. is a schematic of the mechanism of PD1 expression/inhibition.

FIG. 7: Cells stably expressing a Control shRNA (CshRNA) or a MUC1 shRNA (MUC1shRNA) were analyzed for MUC1 and PDL-1 mRNA The indicated A549 (left) and H460 (right) cells were immunoblotted with the indicated antibodies.

Figure: 8: The indicated A549 (left) and H460 (right) cells were immunoblotted with the indicated antibodies.

FIG. 9: Cells stably expressing a Control shRNA (CshRNA) or a MUC1 shRNA (MUC1shRNA) were analyzed for MUC1 and PDL-1 mRNA. The indicated A549 (left) and H460 (right) cells were immunoblotted with the indicated antibodies.

FIG. 10: Cells stably expressing a Control shRNA (CshRNA) or a MUC1 shRNA (MUC1shRNA) were analyzed for MUC1 and PDL-1 mRNA. The indicated A549 (left) and H460 (right) cells were immunoblotted with the indicated antibodies.

FIG. 11: are bar charts. The results are expressed as the relative fold enrichment compared with that obtained for the MUC1 shRNA (assigned a value of 1).

FIG. 12: Cells stably expressing a Control shRNA (CshRNA) or a MUC1 shRNA (MUC1shRNA) were analyzed PDL-1 expression. The results are expressed as the relative luciferase activity obtained for the MUC1 shRNA (assigned a value of 1).

### DETAILED DESCRIPTION OF THE INVENTION

The present invention is based on the discovery that the oncogene Mucin 1 (MUC1) governs PD-L1 expression in tumors.

The PD-L1/PD-1 pathway is a critical mediator of immune escape in malignancy and has emerged as a promising target for immune based therapy. Antibody blockade results in durable disease regression in a subset of patients with chemotherapy resistant disease.

As described herein, it was demonstrated that the oncogene MUC1 governs PD-L1 expression in tumors. Silencing of MUC1 expression using MUC1 specific shRNA or CRISPR results in the near abrogation of PD-L1 expression in multiple myeloma (MM) and acute myeloid leukemia (AML). Exposure to small molecule MUC1 inhibitor similarly decreases PDL1 expression by MM and AML cells rendering them more susceptible CTL mediated lysis.

To elaborate the mechanism by which MUC1 regulates PDL1 expression, we assessed its impact on a family of noncoding RNAs, miR200 that demonstrate homology with PDL1 mRNA. Noncoding RNAs such as microRNAs have been shown to regulate critical aspects of oncogenesis through the selective binding and degradation of mRNAs and modulation of protein expression. It was discovered that that MUC1 silencing in tumor cells leads to 4 fold increase of miR-200c levels. ChIP analysis has furthermore shown that increase in miR-200c expression is achieved via MUC1 chaperoning of SNAIL, a known transcription regulator, to the miR-200c promoter. MiR-200c then in turn is shown to post transcriptionally decrease PD-L1 levels via the binding to the 3′ UTR of PD-L1 in MM cell lines and patient derived tumor cells.

### Mucin-1 Inhibitors

A mucin-1 (MUC1) inhibitor is a compound that decreases expression or activity of MUC1. MUC1 is an oncogenic glycoprotein that is aberrantly expressed in many solid tumor and hematological malignancies including MM. MUC1 plays a vital role in supporting key aspects of the malignant phenotype including cell proliferation and self-renewal, resistance to cytotoxic injury and apoptosis, and capacity for migration and tissue invasion. MUC1 is comprised of an N-terminus that is shed into the circulation and a C-terminus that, upon activation, undergoes homodimerization, translocation to the nucleus and interaction with downstream effectors including Wnt/B catenin, NFKB, and the JAK/STAT pathway.

A MUC1 inhibitor decreases expression or activity of MUC1. A decrease in MUC1 activity is defined by a reduction of a biological function of the MUC1. For example, a decrease or reduction in MUC1 expression or biological activity refers to at least a 1%, 2%, 5%, 10%, 15%, 20%, 25%, 30%, 35%, 40%, 45%, 50%, 60%, 70%, 80%, 90% or 100% decrease in MUC1 expression or activity compared to a control.

A biological activity of a MUC1 inhibitor includes for example upregulation of miR-200c.

MUC1 expression is measured by detecting a MUC1 transcript or protein using standard methods known in the art, such as RT-PCR, microarray, and immunoblotting or immunohistochemistry with MUC1-specific antibodies. For example, a decrease in MUC1 expression refers to at least a 1%, 2%, 5%, 10%, 15%, 20%, 25%, 30%, 35%, 40%, 45%, 50%, 60%, 70%, 80%, 90% or 100% decrease in the level of MUC1 mRNA or MUC1 protein.

The MUC1 inhibitor is an antibody or fragment thereof specific to MUC1. Methods for designing and producing specific antibodies are well-known in the art. In particular embodiments the MUC1 inhibitor is a bi-specific antibody. For example, the bi-specific antibody is specific for MUC1 and A2AR, B7-H3/CD276, B7-H4/VTCN1, BTLA, CD27, CD28, CD40, CD122, CD137, CTLA-4, GITR, ICOS, IDO, KIR, LAG3, OX40, PD1, PD-L1, PD-L2, TIM-3, or VISTA.

The MUC1 inhibitor can also be a small molecule. A “small molecule” as used herein, is meant to refer to a composition that has a molecular weight in the range of less than about 5 kD to 50 daltons, for example less than about 4 kD, less than about 3.5 kD, less than about 3 kD, less than about 2.5 kD, less than about 2 kD, less than about 1.5 kD, less than about 1 kD, less than 750 daltons, less than 500 daltons, less than about 450 daltons, less than about 400 daltons, less than about 350 daltons, less than 300 daltons, less than 250 daltons, less than about 200 daltons, less than about 150 daltons, less than about 100 daltons. Small molecules can be, e.g., nucleic acids, peptides, polypeptides, peptidomimetics, carbohydrates, lipids or other organic or inorganic molecules. Libraries of chemical and/or biological mixtures, such as fungal, bacterial, or algal extracts, are known in the art and can be screened with any of the assays of the invention. For example, the MUC1 inhibitor is G0-203.

Alternatively, the MUC1 inhibitor is for example an antisense MUC1 nucleic acid, a MUC1specific short-interfering RNA, or a MUC1-specific ribozyme. By the term “siRNA” is meant a double stranded RNA molecule which prevents translation of a target mRNA. Standard techniques of introducing siRNA into a cell are used, including those in which DNA is a template from which an siRNA is transcribed. The siRNA includes a sense MUC1 nucleic acid sequence, an anti-sense MUC1nucleic acid sequence or both. Optionally, the siRNA is constructed such that a single transcript has both the sense and complementary antisense sequences from the target gene, e.g., a hairpin (shRNA). Examples of siRNAs and shRNAs are disclosed in the examples herein.

Binding of the siRNA to a MUC1 transcript in the target cell results in a reduction in MUC1 production by the cell. The length of the oligonucleotide is at least 10 nucleotides and may be as long as the naturally-occurring MUC1 transcript. Preferably, the oligonucleotide is 19-25 nucleotides in length. Most preferably, the oligonucleotide is less than 75, 50, 25 nucleotides in length.

### Therapeutic Methods

In various aspects the invention provides method of treating cancer in a subject. The method includes administering to the subject a compound that inhibits the expression or activity of MUC1.

Cells are directly contacted with the compound. Alternatively, the compound is administered systemically.

The subject will receive, has received or is receiving checkpoint inhibitor therapy. The check point inhibitor is administered contemporaneously with MUC1 inhibitor, prior to administration of the MUC1 inhibitor or after administration of the MUC1 inhibitor.

By checkpoint inhibitor it is meant that at the compound inhibits a protein in the checkpoint signally pathway. Proteins in the checkpoint signally pathway include for example, A2AR, B7-H3/CD276, B7-H4/VTCN1, BTLA, CD27, CD28, CD40, CD122, CD137, CTLA-4, GITR, ICOS, IDO, KIR, LAG3, OX40, PD1, PD-L1, PD-L2, TIM-3, or VISTA.

Checkpoint inhibitors are known in the art. For example, the checkpoint inhibitor can be a small molecule. A “small molecule” as used herein, is meant to refer to a composition that has a molecular weight in the range of less than about 5 kD to 50 daltons, for example less than about 4 kD, less than about 3.5 kD, less than about 3 kD, less than about 2.5 kD, less than about 2 kD, less than about 1.5 kD, less than about 1 kD, less than 750 daltons, less than 500 daltons, less than about 450 daltons, less than about 400 daltons, less than about 350 daltons, less than 300 daltons, less than 250 daltons, less than about 200 daltons, less than about 150 daltons, less than about 100 daltons. Small molecules can be, e.g., nucleic acids, peptides, polypeptides, peptidomimetics, carbohydrates, lipids or other organic or inorganic molecules.

Alternatively the checkpoint inhibitor is an antibody is an antibody or fragment thereof. For example, the antibody or fragment thereof is specific to a protein in the checkpoint signaling pathway, such as A2AR, B7-H3/CD276, B7-H4/VTCN1, BTLA, CD27, CD28, CD40, CD122, CD137, CTLA-4, GITR, ICOS, IDO, KIR, LAG3, OX40, PD1, PD-L1, PD-L2, TIM-3, or VISTA.

The subject will receive, has received or is receiving a tumor vaccine consisting of a fusion between autologous dendritic cells (DCs) and tumor cells (DC cell fusions). The DC cell fusions are administered contemporaneously with MUC1 inhibitor, prior to administration of the MUC1 inhibitor or after administration of the MUC1 inhibitor.

Optionally, the patient may receive concurrent treatment with an immunomodulatory agent. These agents include lenalidomide, pomalinomide, or apremilast. Lenalidomide has been shown to boost response to vaccination targeting infectious diseases and in pre-clinical studies enhances T cell response to a DC cell fusion vaccine.

The methods described herein are useful to alleviate the symptoms of a variety of cancers. Any cancer exhibiting chemotherapy resistance or increased PD-L1 expression is suitable for treatment with the methods of the invention. The cancer is a solid tumor or a hematologic tumor. The solid tumor is for example a lung tumor, a breast tumor, or a renal tumor. The hematologic tumor id for example acute myeloid leukemia (AML) or multiple myeloma (MM).

Treatment is efficacious if the treatment leads to clinical benefit such as, a decrease in size, prevalence, or metastatic potential of the tumor in the subject. When treatment is applied prophylactically, “efficacious” means that the treatment retards or prevents tumors from forming or prevents or alleviates a symptom of clinical symptom of the tumor. Efficaciousness is determined in association with any known method for diagnosing or treating the particular tumor type.

### Therapeutic Administration

The invention includes administering to a subject composition comprising a MUC1 inhibitor.

An effective amount of a therapeutic compound is preferably from about 0.1 mg/kg to about 150 mg/kg. Effective doses vary, as recognized by those skilled in the art, depending on route of administration, excipient usage, and co-administration with other therapeutic treatments including use of other anti-proliferative agents or therapeutic agents for treating, preventing or alleviating a symptom of a cancer. A therapeutic regimen is carried out by identifying a mammal, e.g., a human patient suffering from a cancer using standard methods.

Doses may be administered once or more than once. In some embodiments, it is preferred that the therapeutic compound is administered once a week, twice a week, three times a week, four times a week, five times a week, six times a week, or seven times a week for a predetermined duration of time. The predetermined duration of time may be 1 week, 2 weeks, 3 weeks, 4 weeks, 5 weeks, 6 weeks, 7 weeks, 2 months, 3 months, 4 months, 5 months, 6 months, 7 months, 8 months, 9 months, 10 months, 11 months, or up to 1 year.

The pharmaceutical compound is administered to such an individual using methods known in the art. Preferably, the compound is administered orally, rectally, nasally, topically or parenterally, e.g., subcutaneously, intraperitoneally, intramuscularly, and intravenously. The inhibitors are optionally formulated as a component of a cocktail of therapeutic drugs to treat cancers. Examples of formulations suitable for parenteral administration include aqueous solutions of the active agent in an isotonic saline solution, a 5% glucose solution, or another standard pharmaceutically acceptable excipient. Standard solubilizing agents such as PVP or cyclodextrins are also utilized as pharmaceutical excipients for delivery of the therapeutic compounds.

The therapeutic compounds described herein are formulated into compositions for other routes of administration utilizing conventional methods. For example, the therapeutic compounds are formulated in a capsule or a tablet for oral administration. Capsules may contain any standard pharmaceutically acceptable materials such as gelatin or cellulose. Tablets may be formulated in accordance with conventional procedures by compressing mixtures of a therapeutic compound with a solid carrier and a lubricant. Examples of solid carriers include starch and sugar bentonite. The compound is administered in the form of a hard shell tablet or a capsule containing a binder, e.g., lactose or mannitol, conventional filler, and a tableting agent. Other formulations include an ointment, suppository, paste, spray, patch, cream, gel, resorbable sponge, or foam. Such formulations are produced using methods well known in the art.

Therapeutic compounds are effective upon direct contact of the compound with the affected tissue. Accordingly, the compound is administered topically. Alternatively, the therapeutic compounds are administered systemically. For example, the compounds are administered by inhalation. The compounds are delivered in the form of an aerosol spray from pressured container or dispenser which contains a suitable propellant, e.g., a gas such as carbon dioxide, or a nebulizer.

Additionally, compounds are administered by implanting (either directly into an organ or subcutaneously) a solid or resorbable matrix which slowly releases the compound into adjacent and surrounding tissues of the subject.

In some embodiments, it is preferred that the therapeutic compounds described herein are administered in combination with another therapeutic agent, such as a chemotherapeutic agent, radiation therapy, or an anti-mitotic agent. In some aspects, the anti-mitotic agent is administered prior to administration of the present therapeutic compound, in order to induce additional chromosomal instability to increase the efficacy of the present invention to targeting cancer cells. Examples of anti-mitotic agents include taxanes (i.e., paclitaxel, docetaxel), and vinca alkaloids (i.e., vinblastine, vincristine, vindesine, vinorelbine).

### Screening Assays

The invention also provides a method of predicting in vivo expression of PD-L1 by measuring miR-200c levels in serum.

The method includes detecting the expression level of miR-200c in a subject sample, wherein an decrease of expression of miR-200c compared to a normal control cell indicates that the subject's tumor is expressing PD-L1 and would derive a benefit from PD-1 or PDL-1 therapy

### Definitions

The practice of the present invention employs, unless otherwise indicated, conventional techniques of molecular biology, microbiology, cell biology and recombinant DNA, which are within the skill of the art. See, e.g., Sambrook, Fritsch and Maniatis, MOLECULAR CLONING: A LABORATORY MANUAL, 2^{nd} edition (1989); CURRENT PROTOCOLS IN MOLECULAR BIOLOGY (F. M. Ausubel et al. eds., (1987)); the series METHODS IN ENZYMOLOGY (Academic Press, Inc.): PCR 2: A PRACTICAL APPROACH (Mi. MacPherson, B. D. Hames and G. R. Taylor eds. (1995)) and ANIMAL CELL CULTURE (Rd. Freshney, ed. (1987)).

As used herein, certain terms have the following defined meanings. As used in the specification and claims, the singular form “a”, “an”, and “the” include plural references unless the context clearly dictates otherwise. For example, the term “a cell” includes a plurality of cells, including mixtures thereof.

“Treatment” is an intervention performed with the intention of preventing the development or altering the pathology or symptoms of a disorder. Accordingly, “treatment” refers to both therapeutic treatment and prophylactic or preventative measures. Those in need of treatment include those already with the disorder as well as those in which the disorder is to be prevented. In tumor (e.g., cancer) treatment, a therapeutic agent may directly decrease the pathology of tumor cells, or render the tumor cells more susceptible to treatment by other therapeutic agents, e.g., radiation and/or chemotherapy. As used herein, “ameliorated” or “treatment” refers to a symptom which is approaches a normalized value (for example a value obtained in a healthy patient or individual), e.g., is less than 50% different from a normalized value, preferably is less than about 25% different from a normalized value, more preferably, is less than 10% different from a normalized value, and still more preferably, is not significantly different from a normalized value as determined using routine statistical tests.

Thus, treating may include suppressing, inhibiting, preventing, treating, or a combination thereof. Treating refers inter alia to increasing time to sustained progression, expediting remission, inducing remission, augmenting remission, speeding recovery, increasing efficacy of or decreasing resistance to alternative therapeutics, or a combination thereof. “Suppressing” or “inhibiting”, refers inter alia to delaying the onset of symptoms, preventing relapse to a disease, decreasing the number or frequency of relapse episodes, increasing latency between symptomatic episodes, reducing the severity of symptoms, reducing the severity of an acute episode, reducing the number of symptoms, reducing the incidence of disease-related symptoms, reducing the latency of symptoms, ameliorating symptoms, reducing secondary symptoms, reducing secondary infections, prolonging patient survival, or a combination thereof. The symptoms are primary, while in another embodiment, symptoms are secondary. “Primary” refers to a symptom that is a direct result of the proliferative disorder, while, secondary refers to a symptom that is derived from or consequent to a primary cause. Symptoms may be any manifestation of a disease or pathological condition.

The “treatment of cancer or tumor cells”, refers to an amount of peptide or nucleic acid, described throughout the specification, capable of invoking one or more of the following effects: (1) inhibition of tumor growth, including, (i) slowing down and (ii) complete growth arrest; (2) reduction in the number of tumor cells; (3) maintaining tumor size; (4) reduction in tumor size; (5) inhibition, including (i) reduction, (ii) slowing down or (iii) complete prevention, of tumor cell infiltration into peripheral organs; (6) inhibition, including (i) reduction, (ii) slowing down or (iii) complete prevention, of metastasis; (7) enhancement of anti-tumor immune response, which may result in (i) maintaining tumor size, (ii) reducing tumor size, (iii) slowing the growth of a tumor, (iv) reducing, slowing or preventing invasion and/or (8) relief, to some extent, of the severity or number of one or more symptoms associated with the disorder.

As used herein, “an ameliorated symptom” or “treated symptom” refers to a symptom which approaches a normalized value, e.g., is less than 50% different from a normalized value, preferably is less than about 25% different from a normalized value, more preferably, is less than 10% different from a normalized value, and still more preferably, is not significantly different from a normalized value as determined using routine statistical tests.

The terms “patient” or “individual” are used interchangeably herein, and refers to a mammalian subject to be treated, with human patients being preferred. In some cases, the methods of the invention find use in experimental animals, in veterinary application, and in the development of animal models for disease, including, but not limited to, rodents including mice, rats, and hamsters; and primates.

By the term “modulate,” it is meant that any of the mentioned activities, are, e.g., increased, enhanced, increased, augmented, agonized (acts as an agonist), promoted, decreased, reduced, suppressed blocked, or antagonized (acts as an antagonist). Modulation can increase activity more than 1-fold, 2-fold, 3-fold, 5-fold, 10-fold, 100-fold, etc., over baseline values. Modulation can also decrease its activity below baseline values.

As used herein, the term “administering to a cell” (e.g., an expression vector, nucleic acid, a delivery vehicle, agent, and the like) refers to transducing, transfecting, microinjecting, electroporating, or shooting, the cell with the molecule. In some aspects, molecules are introduced into a target cell by contacting the target cell with a delivery cell (e.g., by cell fusion or by lysing the delivery cell when it is in proximity to the target cell).

Dendritic cells (DCs) are potent APCs. DCs are minor constituents of various immune organs such as spleen, thymus, lymph node, epidermis, and peripheral blood. For instance, DCs represent merely about 1% of crude spleen (see Steinman et al. (1979) J. Exp. Med 149: 1) or epidermal cell suspensions (see Schuler et al. (1985) J. Exp. Med 161:526; Romani et al. J. Invest. Dermatol (1989) 93: 600) and 0.1-1% of mononuclear cells in peripheral blood (see Freudenthal et al. Proc. Natl Acad Sci USA (1990) 87: 7698). Methods for isolating DCs from peripheral blood or bone marrow progenitors are known in the art. (See Inaba et al. (1992) J. Exp. Med 175:1157; Inaba et al. (1992) J. Exp, Med 176: 1693-1702; Romani et al. (1994) J. Exp. Med. 180: 83-93; Sallusto et al. (1994) J. Exp. Med 179: 1109-1118)). Preferred methods for isolation and culturing of DCs are described in Bender et al. (1996) J. Immun. Meth. 196:121-135 and Romani et al. (1996) J. Immun. Meth 196:137-151.

Thus, the term “cytokine” refers to any of the numerous factors that exert a variety of effects on cells, for example, inducing growth or proliferation. Non-limiting examples of cytokines include, IL-2, stem cell factor (SCF), IL-3, IL-6, IL-7, IL-12, IL-15, G-CSF, GM-CSF, IL-1 α, IL-1 β, MIP-1 α, LIF, c-kit ligand, TPO, and flt3 ligand. Cytokines are commercially available from several vendors such as, for example, Genzyme Corp. (Framingham, Mass.), Genentech (South San Francisco, Calif.), Amgen (Thousand Oaks, Calif.) and Immunex (Seattle, Wash.). It is intended, although not always explicitly stated, that molecules having similar biological activity as wild-type or purified cytokines (e.g., recombinantly produced cytokines) are intended to be used within the spirit and scope of the invention and therefore are substitutes for wild-type or purified cytokines.

“Costimulatory molecules” are involved in the interaction between receptor-ligand pairs expressed on the surface of antigen presenting cells and T cells. One exemplary receptor-ligand pair is the B7 co-stimulatory molecules on the surface of DCs and its counter-receptor CD28 or CTLA-4 on T cells. (See Freeman et al. (1993) Science 262:909-911; Young et al. (1992) J. Clin. Invest 90: 229; Nabavi et al. Nature 360:266)). Other important costimulatory molecules include, for example, CD40, CD54, CD80, and CD86. These are commercially available from vendors identified above.

A “hybrid” cell refers to a cell having both antigen presenting capability and also expresses one or more specific antigens. In one embodiment, these hybrid cells are formed by fusing, in vitro, APCs with cells that are known to express the one or more antigens of interest. As used herein, the term “hybrid” cell and “fusion” cell are used interchangeably.

A “control” cell refers to a cell that does not express the same antigens as the population of antigen-expressing cells.

The term “culturing” refers to the in vitro propagation of cells or organisms on or in media of various kinds, it is understood that the descendants 30 of a cell grown in culture may not be completely identical (i.e., morphologically, genetically, or phenotypically) to the parent cell. By “expanded” is meant any proliferation or division of cells.

An “effective amount” is an amount sufficient to effect beneficial or desired results. An effective amount can be administered in one or more administrations, applications or dosages. For purposes of this invention, an effective amount of hybrid cells is that amount which promotes expansion of the antigenic-specific immune effector cells, e.g., T cells.

An “isolated” population of cells is “substantially free” of cells and materials with which it is associated in nature. By “substantially free” or “substantially pure” is meant at least 50% of the population is the desired cell type, preferably at least 70%, more preferably at least 80%, and even more preferably at least 90%. An “enriched” population of cells is at least 5% fused cells. Preferably, the enriched population contains at least 10%, more preferably at least 20%, and most preferably at least 25% fused cells.

The term “autogeneic”, or “autologous”, as used herein, indicates the origin of a cell. Thus, a cell being administered to an individual (the “recipient”) is autogeneic if the cell was derived from that individual (the “donor”) or a genetically identical individual (i.e., an identical twin of the individual). An autogeneic cell can also be a progeny of an autogeneic cell. The term also indicates that cells of different cell types are derived from the same donor or genetically identical donors. Thus, an effector cell and an antigen presenting cell are said to be autogeneic if they were derived from the same donor or from an individual genetically identical to the donor, or if they are progeny of cells derived from the same donor or from an individual genetically identical to the donor.

Similarly, the term “allogeneic”, as used herein, indicates the origin of a cell. Thus, a cell being administered to an individual (the “recipient”) is allogeneic if the cell was derived from an individual not genetically identical to the recipient. In particular, the term relates to non-identity in expressed MHC molecules. An allogeneic cell can also be a progeny of an allogeneic cell. The term also indicates that cells of different cell types are derived from genetically non-identical donors, or if they are progeny of cells derived from genetically non-identical donors. For example, an APC is said to be allogeneic to an effector cell if they are derived from genetically non-identical donors.

A “subject” is a vertebrate, preferably a mammal, more preferably a human. Mammals include, but are not limited to, murines, simians, humans, farm animals, sport animals, and pets.

As used herein, “genetic modification” refers to any addition, deletion or disruption to a cell's endogenous nucleotides.

A “viral vector” is defined as a recombinantly produced virus or viral particle that comprises a polynucleotide to be delivered into a host cell, either in vivo, ex vivo or in vitro. Examples of viral vectors include retroviral vectors, adenovirus vectors, adeno-associated virus vectors and the like. In aspects where gene transfer is mediated by a retroviral vector, a vector construct refers to the polynucleotide comprising the retroviral genome or part thereof, and a therapeutic gene.

As used herein, the terms “retroviral mediated gene transfer” or “retroviral transduction” carries the same meaning and refers to the process by which a gene or a nucleic acid sequence is stably transferred into the host cell by virtue of the virus entering the cell and integrating its genome into the host cell genome. The virus can enter the host cell via its normal mechanism of infection or be modified such that it binds to a different host cell surface receptor or ligand to enter the cell.

Retroviruses carry their genetic information in the form of RNA. However, once the virus infects a cell, the RNA is reverse-transcribed into the DNA form that integrates into the genomic DNA of the infected cell. The integrated DNA form is called a provirus.

In aspects where gene transfer is mediated by a DNA viral vector, such as a adenovirus (Ad) or adeno-associated virus (AAV), a vector construct refers to the polynucleotide comprising the viral genome or part thereof, and a therapeutic gene. Adenoviruses (Ads) are a relatively well characterized, homogenous group of viruses, including over 50 serotypes. (See, e.g., WO 95/27071). Ads are easy to grow and do not integrate into the host cell genome. Recombinant Ad-derived vectors, particularly those that reduce the potential for recombination and generation of wild-type virus, have also been constructed. (See, WO 95/00655; WO 95/11984). Wild-type AAV has high infectivity and specificity integrating into the host cells genome. (See Hermonat and Muzyczka (1984) PNAS USA 81:6466-6470; Lebkowski et al., (1988) Mol Cell Biol 8:3988-3996).

Vectors that contain both a promoter and a cloning site into which a polynucleotide can be operatively linked are well known in the art. Such vectors are capable of transcribing RNA in vitro or in vivo, and are commercially available from sources such as Stratagene (La Jolla, Calif.) and Promega Biotech (Madison, Wis.). In order to optimize expression and/or in vitro transcription, it may be necessary to remove, add or alter 5′ and/or 3′ untranslated portions of the clones to eliminate extra, potential inappropriate alternative translation initiation codons or other sequences that may interfere with or reduce expression, either at the level of transcription or translation. Alternatively, consensus ribosome binding sites can be inserted immediately 5′ of the start codon to enhance expression. Examples of suitable vectors are viruses, such as baculovirus and retrovirus, bacteriophage, cosmid, plasmid, fungal vectors and other recombination vehicles typically used in the art which have been described for expression in a variety of eukaryotic and prokaryotic hosts, and may be used for gene therapy as well as for simple protein expression.

Among these are several non-viral vectors, including DNA/liposome complexes, and targeted viral protein DNA complexes. To enhance delivery to a cell, the nucleic acid or proteins of this invention can be conjugated to antibodies or binding fragments thereof which bind cell surface antigens, e.g., TCR, CD3 or CD4. Liposomes that also comprise a targeting antibody or fragment thereof can be used in the methods of this invention. This invention also provides the targeting complexes for use in the methods disclosed herein.

Polynucleotides are inserted into vector genomes using methods well known in the art. For example, insert and vector DNA can be contacted, under suitable conditions, with a restriction enzyme to create complementary ends on each molecule that can pair with each other and be joined together with a ligase. Alternatively, synthetic nucleic acid linkers can be ligated to the termini of restricted polynucleotide. These synthetic linkers contain nucleic acid sequences that correspond to a particular restriction site in the vector DNA. Additionally, an oligonucleotide containing a termination codon and an appropriate restriction site can be ligated for insertion into a vector containing, for example, some or all of the following: a selectable marker gene, such as the neomycin gene for selection of stable or transient transfectants in mammalian cells; enhancer/promoter sequences from the immediate early gene of human CMV for high levels of transcription; transcription termination and RNA processing signals from SV40 for mRNA stability; SV40 polyoma origins of replication and ColEI for proper episomal replication; versatile multiple cloning sites; and T7 and SP6 RNA promoters for in vitro transcription of sense and antisense RNA. Other means are well known and available in the art.

As used herein, “expression” refers to the process by which polynucleotides are transcribed into mRNA and translated into peptides, polypeptides, or proteins. If the polynucleotide is derived from genomic DNA, expression may include splicing of the mRNA, if an appropriate eukaryotic host is selected. Regulatory elements required for expression include promoter sequences to bind RNA polymerase and transcription initiation sequences for ribosome binding. For example, a bacterial expression vector includes a promoter such as the lac promoter and for transcription initiation the Shine-Dalgarno sequence and the start codon AUG (Sambrook et al. (1989), supra). Similarly, a eukaryotic expression vector includes a heterologous or homologous promoter for RNA polymerase II, a downstream polyadenylation signal, the start codon AUG, and a termination codon for detachment of the ribosome. Such vectors can be obtained commercially or assembled by the sequences described in methods well known in the art, for example, the methods described above for constructing vectors in general.

The terms “major histocompatibility complex” or “MHC” refers to a complex of genes encoding cell-surface molecules that are required for antigen presentation to immune effector cells such as T cells and for rapid graft rejection. In humans, the MHC complex is also known as the HLA complex. The proteins encoded by the MHC complex are known as “MHC molecules” and are classified into class I and class II MHC molecules. Class I MHC molecules include membrane heterodimeric proteins made up of an α chain encoded in the MHC associated non-covalently with β2-microglobulin. Class I MHC molecules are expressed by nearly all nucleated cells and have been shown to function in antigen presentation to CD8+ T cells. Class I molecules include HLA-A, -B, and -C in humans. Class II MHC molecules also include membrane heterodimeric proteins consisting of non-covalently associated and J3 chains. Class II MHCs are known to function in CD4+ T cells and, in humans, include HLA-DP, -DQ, and DR. The term “MHC restriction” refers to a characteristic of T cells that permits them to recognize antigen only after it is processed and the resulting antigenic peptides are displayed in association with either a class I or class II MHC molecule. Methods of identifying and comparing MHC are well known in the art and are described in Allen M. et al. (1994) Human Imm. 40:25-32; Santamaria P. et al. (1993) Human Imm. 37:39-50; and Hurley C. K. et al. (1997) Tissue Antigens 50:401-415.

The term “sequence motif” refers to a pattern present in a group of 15 molecules (e.g., amino acids or nucleotides). For instance, in one embodiment, the present invention provides for identification of a sequence motif among peptides present in an antigen. In this embodiment, a typical pattern may be identified by characteristic amino acid residues, such as hydrophobic, hydrophilic, basic, acidic, and the like.

The term “peptide” is used in its broadest sense to refer to a compound of two or more subunit amino acids, amino acid analogs, or peptidomimetics. The subunits may be linked by peptide bonds. In another embodiment, the subunit may be linked by other bonds, e.g. ester, ether, etc.

As used herein the term “amino acid” refers to either natural and/or 25 unnatural or synthetic amino acids, including glycine and both the D or L optical isomers, and amino acid analogs and peptidomimetics. A peptide of three or more amino acids is commonly called an oligopeptide if the peptide chain is short. If the peptide chain is long, the peptide is commonly called a polypeptide or a protein.

As used herein, “solid phase support” is used as an example of a “carrier” and is not limited to a specific type of support. Rather a large number of supports are available and are known to one of ordinary skill in the art. Solid phase supports include silica gels, resins, derivatized plastic films, glass beads, cotton, plastic beads, and alumina gels. A suitable solid phase support may be selected on the basis of desired end use and suitability for various synthetic protocols. For example, for peptide synthesis, solid phase support may refer to resins such as polystyrene (e.g., PAM-resin obtained from Bachem Inc., Peninsula Laboratories, etc.), POLYHIPE® resin (obtained from Aminotech, Canada), polyamide resin (obtained from Peninsula Laboratories), polystyrene resin grafted with polyethylene glycol (TentaGel®, Rapp Polymere, Tubingen, Germany) or polydimethylacrylamide resin (obtained from MilligenlBiosearch, Calif.). In a preferred embodiment for peptide synthesis, solid phase support refers to polydimethylacrylamide resin.

The term “aberrantly expressed” refers to polynucleotide sequences in a cell or tissue which are differentially expressed (either over-expressed or under-expressed) when compared to a different cell or tissue whether or not of the same tissue type, i.e., lung tissue versus lung cancer tissue.

“Host cell” or “recipient cell” is intended to include any individual cell or cell culture which can be or have been recipients for vectors or the incorporation of exogenous nucleic acid molecules, polynucleotides and/or proteins. It also is intended to include progeny of a single cell, and the progeny may not necessarily be completely identical (in morphology or in genomic or total DNA complement) to the original parent cell due to natural, accidental, or deliberate mutation. The cells may be prokaryotic or eukaryotic, and include but are not limited to bacterial cells, yeast cells, animal cells, and mammalian cells, e.g., murine, rat, simian or human.

An “antibody” is an immunoglobulin molecule capable of binding an antigen. As used herein, the term encompasses not only intact immunoglobulin molecules, but also anti-idiotypic antibodies, mutants, fragments, fusion proteins, humanized proteins and modifications of the immunoglobulin molecule that comprise an antigen recognition site of the required specificity.

An “antibody complex” is the combination of antibody and its binding partner or ligand.

A “native antigen” is a polypeptide, protein or a fragment containing an epitope, which induces an immune response in the subject.

The term “isolated” means separated from constituents, cellular and otherwise, in which the polynucleotide, peptide, polypeptide, protein, antibody, or fragments thereof, are normally associated with in nature. As is apparent to those of skill in the art, a non-naturally occurring polynucleotide, peptide, polypeptide, protein, antibody, or fragments thereof, does not require “isolation” to distinguish it from its naturally occurring counterpart. In addition, a “concentrated”, “separated” or “diluted” polynucleotide, peptide, polypeptide, protein, antibody, or fragments thereof, is distinguishable from its naturally occurring counterpart in that the concentration or number of molecules per volume is greater than “concentrated” or less than “separated” than that of its naturally occurring counterpart. A polynucleotide, peptide, polypeptide, protein, antibody, or fragments thereof, which differs from the naturally occurring counterpart in its primary sequence or for example, by its glycosylation pattern, need not be present in its isolated form since it is distinguishable from its naturally occurring counterpart by its primary sequence, or alternatively, by another characteristic such as glycosylation pattern. Although not explicitly stated for each of the inventions disclosed herein, it is to be understood that all of the above embodiments for each of the compositions disclosed below and under the appropriate conditions, are provided by this invention. Thus, a non-naturally occurring polynucleotide is provided as a separate embodiment from the isolated naturally occurring polynucleotide. A protein produced in a bacterial cell is provided as a separate embodiment from the naturally occurring protein isolated from a eukaryotic cell in which it is produced in nature.

A “composition” is intended to mean a combination of active agent and another compound or composition, inert (for example, a detectable agent, carrier, solid support or label) or active, such as an adjuvant.

A “pharmaceutical composition” is intended to include the combination of an active agent with a carrier, inert or active, making the composition suitable for diagnostic or therapeutic use in vitro, in vivo or ex vivo.

As used herein, the term “pharmaceutically acceptable carrier” encompasses any of the standard pharmaceutical carriers, such as a phosphate buffered saline solution, water, and emulsions, such as an oil/water or water/oil emulsion, and various types of wetting agents. The compositions also can include stabilizers and preservatives. For examples of carriers, stabilizers and adjuvants, see Martin, REMINGTON'S PHARM. SCI, 15th Ed. (Mack Publ. Co., Easton (1975)).

As used herein, the term “inducing an immune response in a subject” is a term well understood in the art and intends that an increase of at least about 2-fold, more preferably at least about 5-fold, more preferably at least about 10-fold, more preferably at least about 100-fold, even more preferably at least about 500-fold, even more preferably at least about 1000-fold or more in an immune response to an antigen (or epitope) can be detected (measured), after introducing the antigen (or epitope) into the subject, relative to the immune response (if any) before introduction of the antigen (or epitope) into the subject. An immune response to an antigen (or epitope), includes, but is not limited to, production of an antigen-specific (or epitope-specific) antibody, and production of an immune cell expressing on its surface a molecule which specifically binds to an antigen (or epitope). Methods of determining whether an immune response to a given antigen (or epitope) has been induced are well known in the art. For example, antigen specific antibody can be detected using any of a variety of immunoassays known in the art, including, but not limited to, ELISA, wherein, for example, binding of an antibody in a sample to an immobilized antigen (or epitope) is detected with a detectably-labeled second antibody (e.g., enzyme-labeled mouse anti-human Ig antibody). Immune effector cells specific for the antigen can be detected any of a variety of assays known to those skilled in the art, including, but not limited to, FACS, or, in the case of CTLs, ⁵¹CR-release assays, or ³H-thymidine uptake assays.

By substantially free of endotoxin is meant that there is less endotoxin per dose of cell fusions than is allowed by the FDA for a biologic, which is a total endotoxin of 5 EU/kg body weight per day.

By substantially free for mycoplasma and microbial contamination is meant as negative readings for the generally accepted tests know to those skilled in the art. For example, mycoplasm contamination is determined by sub-culturing a cell sample in broth medium and distributed over agar plates on day 1, 3, 7, and 14 at 37° C. with appropriate positive and negative controls. The product sample appearance is compared microscopically, at 100×, to that of the positive and negative control. Additionally, inoculation of an indicator cell culture is incubated for 3 and 5 days and examined at 600× for the presence of mycoplasmas by epifluorescence microscopy using a DNA-binding fluorochrome. The product is considered satisfactory if the agar and/or the broth media procedure and the indicator cell culture procedure show no evidence of mycoplasma contamination.

The sterility test to establish that the product is free of microbial contamination is based on the U.S. Pharmacopedia Direct Transfer Method. This procedure requires that a pre-harvest medium effluent and a pre-concentrated sample be inoculated into a tube containing tryptic soy broth media and fluid thioglycollate media. These tubes are observed periodically for a cloudy appearance (turpidity) for a fourteen day incubation. A cloudy appearance on any day in either medium indicate contamination, with a clear appearance (no growth) testing substantially free of contamination.

### EXAMPLES

### Example: General Methods

### Preparation of Multiple Myeloma Cell Lines

Human multiple myeloma cell lines RPMI-8226 and U266 cells were obtained from ATCC. Cell lines were cultured in RPMI 1640 medium containing 10% heat inactivated FBS, 2 mM/L L-glutamine, 100 U/ml penicillin, and 100 mg/ml streptomycin. The cell lines were transduced with a lentiviral vector expressing either MUC1 shRNA (Sigma) or a scrambled control shRNA (CshRNA, sigma) vectors in presence of 4-8 ug/ml polybrene (sigma). Transduced cells were selected using puromycin (2 μg/ml). Alternatively, RPMI and U266 cancer cells were stably transduced with a lentiviral vector expressing miR-200c with a GFP selection marker or pHR-GFP (control). Transduced cells were selected by flow cytometric sorting for GFP positive cells. RPMI and U266 cells were also treated with the MUC1-C inhibitor peptide GO-203 (2.5 uM) and a control peptide (CP-2).

### Immunoblotting

Cells were lysed using NP-40 lysis buffer containing protease inhibitor cocktail (Thermo scientific). Soluble proteins were subjected to immunoblotting with anti-MUC1-C (LabVision), anti-PDL1 (Cell Signaling Technology), anti-SNAIL (Santa Cruz Biotechnology), and anti-Beta-Actin (Sigma) antibodies. Detection of immune complex was achieved using horseradish peroxidase-conjugated secondary antibodies and enhanced chemiluminescence (GE Healthcare) detection system.

### FACS Analysis

RPMI and U266 cells were analyzed for MUC1 expression and PD-L1 expression by multichannel flow cytometric analysis. Cells were incubated with monoclonal antibody (mAb) DF3 (anti-MUC1-N), anti PD-L1 (Cell signaling) or a control mouse IgG1 for 30 minutes, followed by secondary labeling of the cells with phycoerythrin (PE)-conjugated goat anti-mouse IgG for an additional 30 minutes. The cells were then fixed in 2% paraformaldehyde. Stained cells were analyzed by flow cytometry using FACScan and CellQuest Pro software (BD Biosciences).

### Quantitative RT-PCR

For qRT-PCR, complementary DNA (cDNA) synthesis was performed with 1 μg of total RNA using the Thermoscript RT-PCR system (Invitrogen). The SYBR green qPCR assay kit (Applied Biosystems) was used with 1 μl of diluted cDNA for each sample and amplified with the ABI prism 7000 sequence detector (ABI). The forward and reverse primers for qPCR of MUC1, PDL1 and GAPDH are listed in supplementary table S1. Statistical significant was determined by the student t test.

### CTL Assay

Lysis of MM cells by allogeneic T cells following MUC1 downregulation was assessed in a standard CTL flourochrome assay.

### Analysis of miR-200c Expression

Total RNA was isolated from cells using the RNeasy total RNA isolation kit (Qiagen). cDNAs were prepared from lug of total RNA using the cDNA synthesis kit specific for small RNA (System Biosciences). Expression of miR-200c was assessed by qPCR with a universal reverse primer and forward primers specific for miR-200c. Human U6 small RNA was used as control. For qPCR, the SYBR green qPCR assay kit (Applied Biosystems) was used with 1 ul of diluted cDNA sample and analyzed with the ABI Prism 7000 Sequence Detector (Applied Biosystems). Fold enrichment was calculated as described [Wang Q, Mol. Cell, 2005].

### PDL-1 3′UTR Reporter Assay

Cells cultured in 6-well plates were either transfected with an empty vector or PDL1-3′UTR reporter (Active Motif) containing a miR-200c binding site. Plasmids were transfected with cells in presence of superfect transfection reagent (Qiagen) and incubated for another 48 hours. At the end of incubation period, cells were lysed with lysis-substrate buffer supplied in the kit (Active Motif) from vendor and were analyzed using the dual luciferase assay kit (Promega).

### ChIP Assay

Soluble chromatin was prepared as previously described and immune-precipitated with anti-SNAIL or a control non-immune immunoglobulin IgG. For real time ChIP qPCR, 2 μl from a 50 μl DNA extraction were used with the SYBR green master mix (Applied Biosystems) and the samples were amplified with the ABI Prism 7000 Sequence Detector (Applied Biosystems). The primers used for ChIP-qPCR for the PDL1 and GAPDH promoter as the control region are listed in supplementary table SII. The relative fold enrichment was calculated as described [Wang Q, Mol. Cell, 2005].

### Example 2: MUC1 Oncoprotein Regulates PD-L1 Expression In Multiple Myeloma Cells

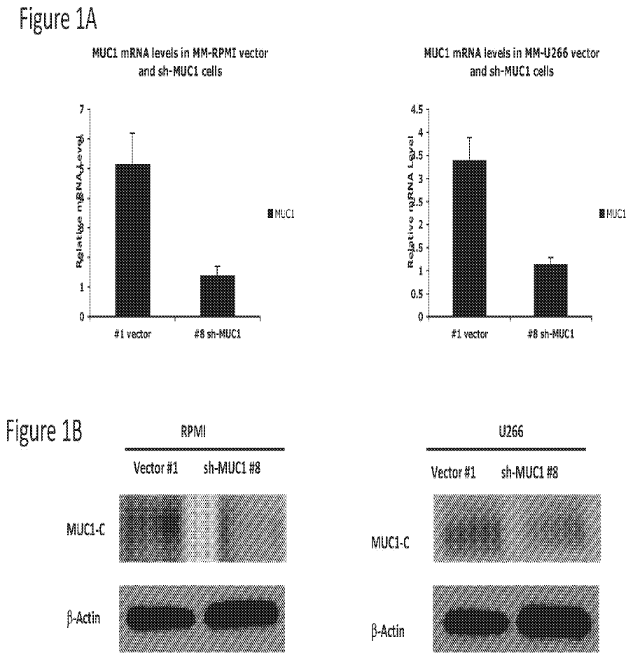

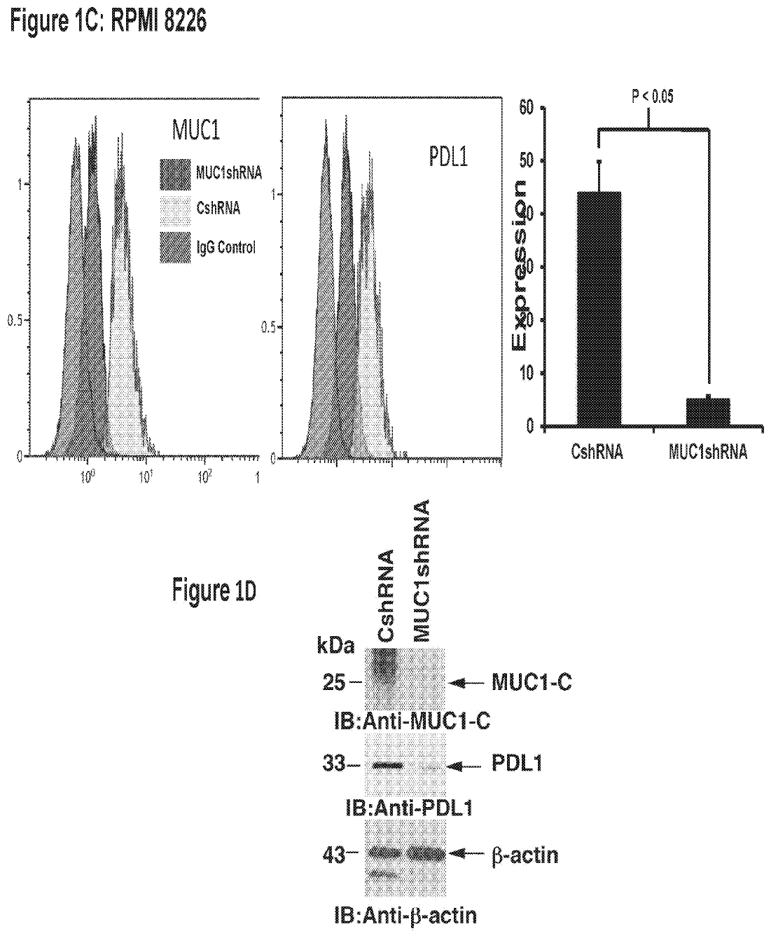

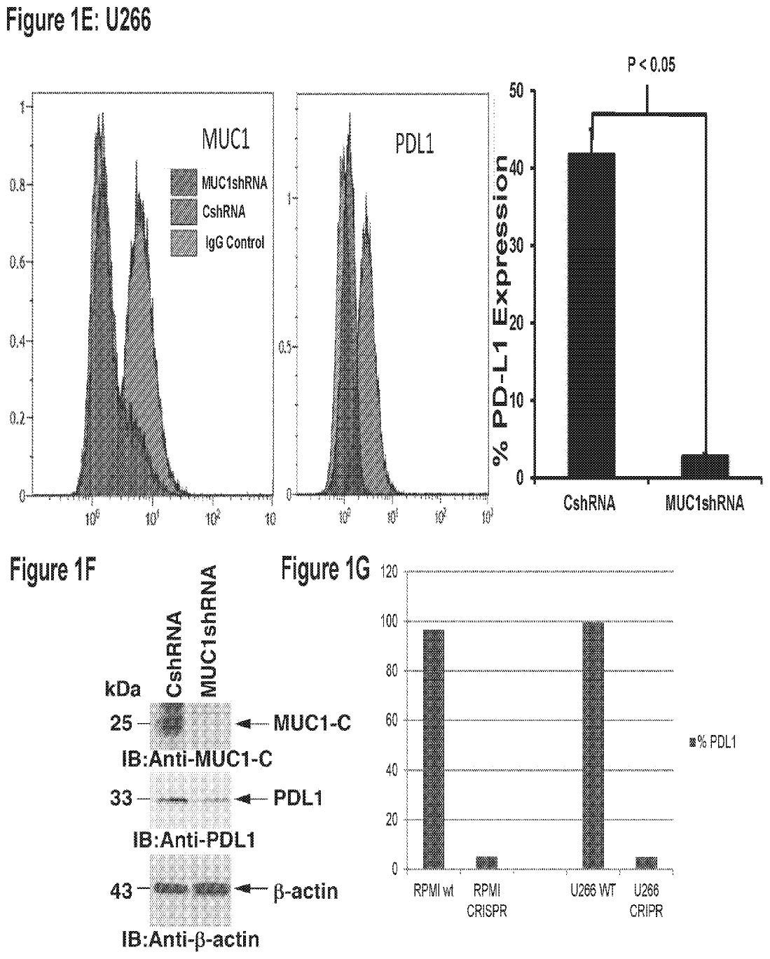

Multiple myeloma cell lines RPMI 8226 and U266 exhibit high levels of MUC1 and PDL-1 expression as determined by flow cytometric analysis (FIG. 1A). To assess the role of MUC1 in the regulation of PD-L1 expression, MUC1 expression was silenced in RPMI 8226 and U266 human myeloma cell lines via lentiviral transfection with MUC1 specific shRNA (FIG. 1B). Inhibition of MUC1 expression was associated with the dramatic reduction in levels of PD-L1 by RPMI cells as determined by bi-dimensional flow cytometry. Reduction of PD-L1 expression following lentiviral transfection with MUC1 specific shRNA was confirmed by western blot analysis. (FIG. 1C, left). The effect of inhibition of MUC1 on PD-L1 expression was confirmed using CRISPR technology altering the genetic sequence encoding the MUC1-C subunit. Intriguingly, levels of PD-L1 mRNA were unchanged in RPMI cells silenced for MUC1-C expression (data not shown) consistent with the post-transcriptional regulation of the PD-L1 protein.

The effect of MUC1 signaling on PD-L1 was next interrogated in primary MM cells isolated from bone marrow aspirates obtained from patients with active disease. GO-203 is a cell penetrating peptide bearing a CQC motif that intercalates with the MUC1-C subunit at the plasma membrane and prevents homodimerization necessary for nuclear translocation and downstream signaling. Of note, GO-203 does not alter MUC11 expression. In vitro exposure of myeloma cell lines and primary myeloma cells to sub-lethal doses of GO-203 resulted in a dose dependent decrease in PD-L1 expression. These findings in aggregate demonstrate that the MUC1 oncogene is a critical mediator of PD-L1 expression via MUC1 signaling and interaction with downstream effectors.

### Example 3: PD-L1 3′UTR Has A miR-200c Binding Site And Respond To miR-200c Over Expression In Multiple Myeloma

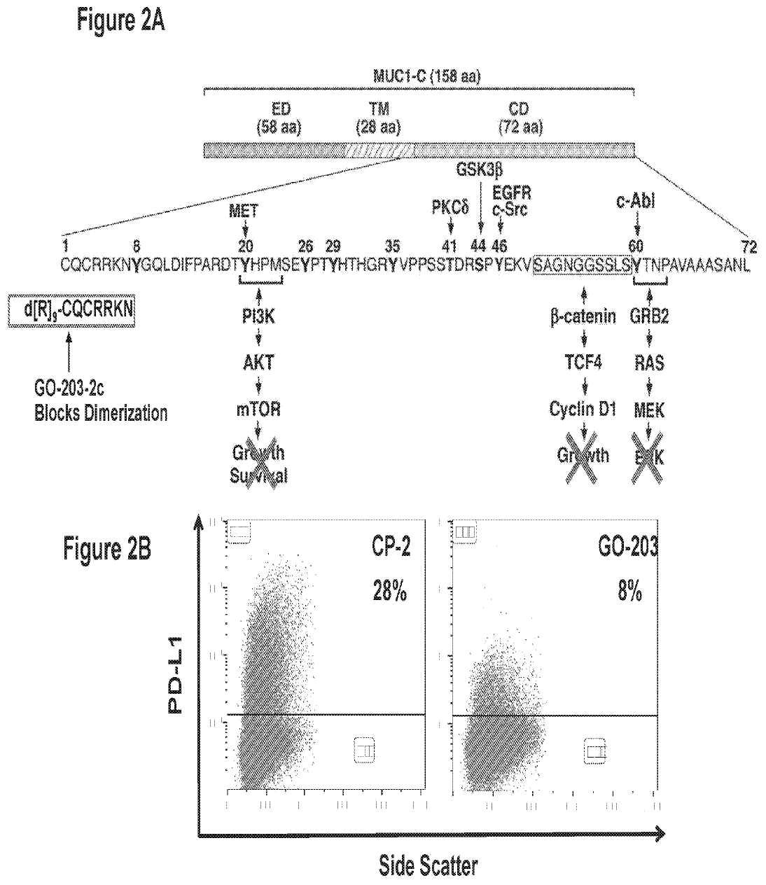

There has been increasing understanding of the vital role that noncoding RNAs play in the regulation of cell signaling and as a mediator of critical aspects of oncogenesis. Micro-RNAs regulate expression of target genes by direct binding to the 3′UTR of the candidate mRNA preventing translation and protein production. A sequence analysis of PD-L1 3′UTR revealed the presence of a miR-200c binding site (FIG. 2A). The interaction between miR-200c and PD-L1 was recently noted in a lung cancer model. It has been previously demonstrated in a solid tumor model that MUC1 regulates miR-200c expression. We subsequently confirmed the interaction of miR200c and PDL1 mRNA in the MM model. Using chromatin immunopreciptation (ChIP) analysis of RPMI cells binding of miR200c and PDL1 mRNA was confirmed.

We subsequently examined the effect of miR-200c on PD-L1 expression in MM cells. Using a lentiviral vector with GFP tags, the miR-200c expressing viral particles were transduced into RPMI cells. A marked decrease in the expression of PDL-1 proteins in GFP positive cells was noted by bidimensional flow cytometric analysis (FIG. 2B). In addition, following flow cytometric sorting of the GFP+ population, protein analysis by western immunblot of the whole cell lysates from these cells revealed a clear decrease in expression of PDL-1 proteins in the miR-200c high population (FIG. 2C). To extend this analysis, similar studies were performed on U266 multiple myeloma cells. Ectopic expression of miR-200c in U266 cells resulted in a decrease in the expression of PD-L1 by FACS (FIG. 2D) and by western blot (FIG. 2E). To examine the effect of MUC1 expression on miR200c levels in MM, the effect of silencing MUC1 via lentiviral transfection of RPMI and U266 cells with MUC1 specific shRNA was assessed. Downregulation of MUC1 expression resulted in a corresponding increase in miR200c levels that was not observed following lentiviral transfection with a control vector.

### Example 4: MUC1-C Suppresses The MicroRNA miR-200c By A SNAIL Dependent Mechanism

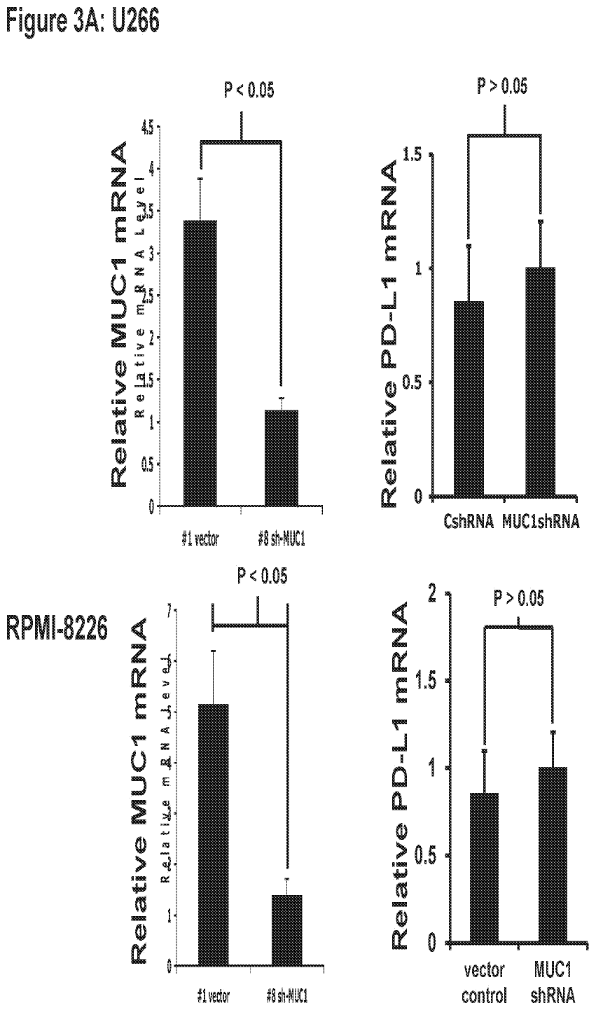

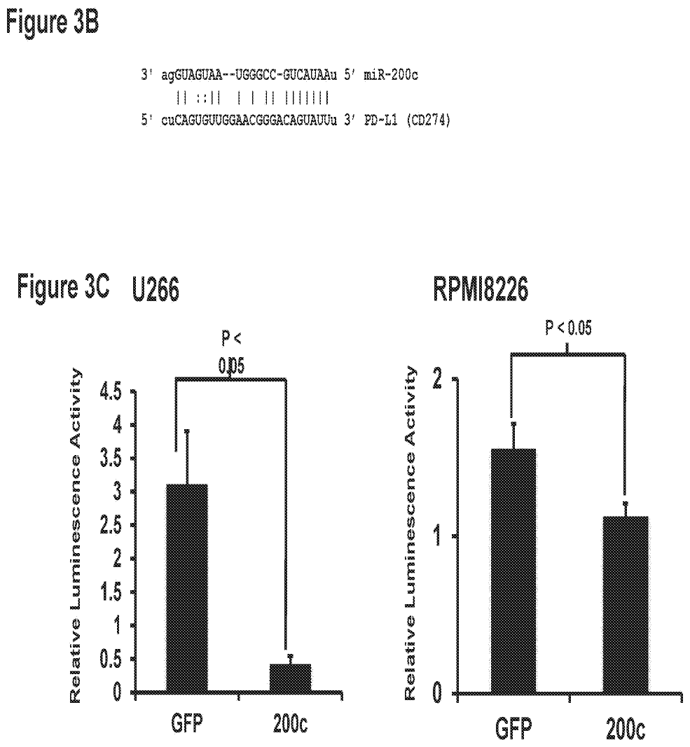

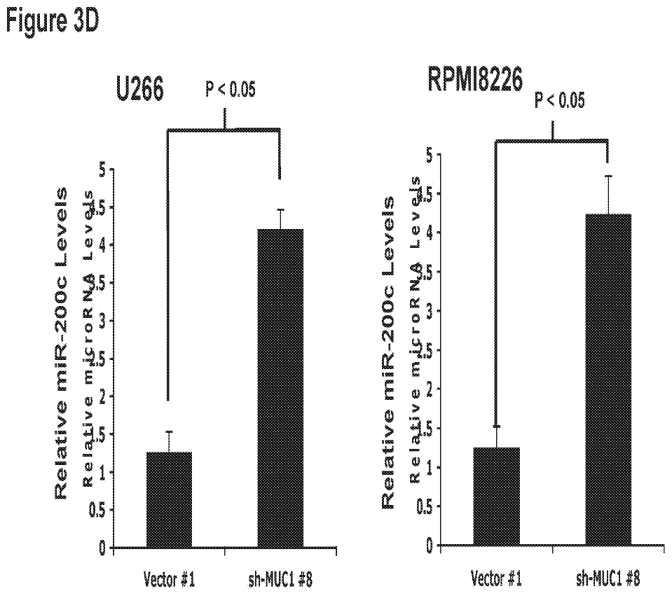

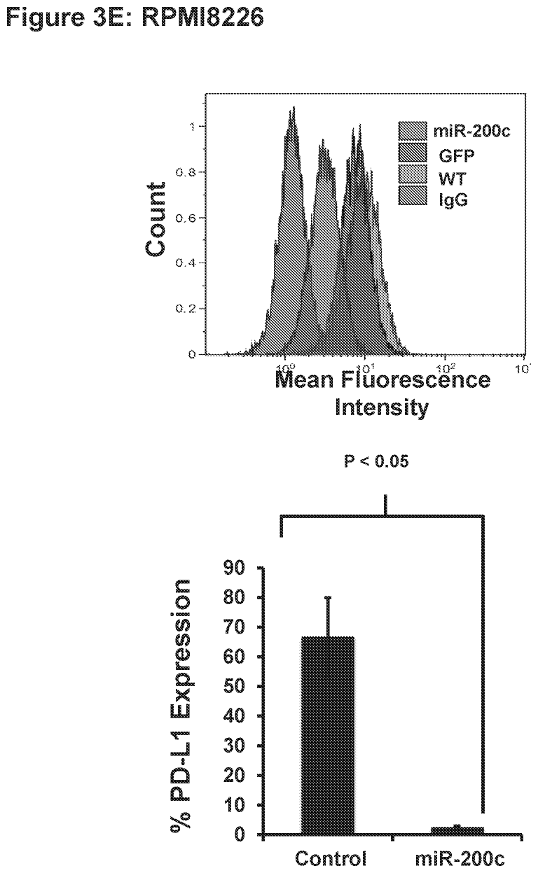

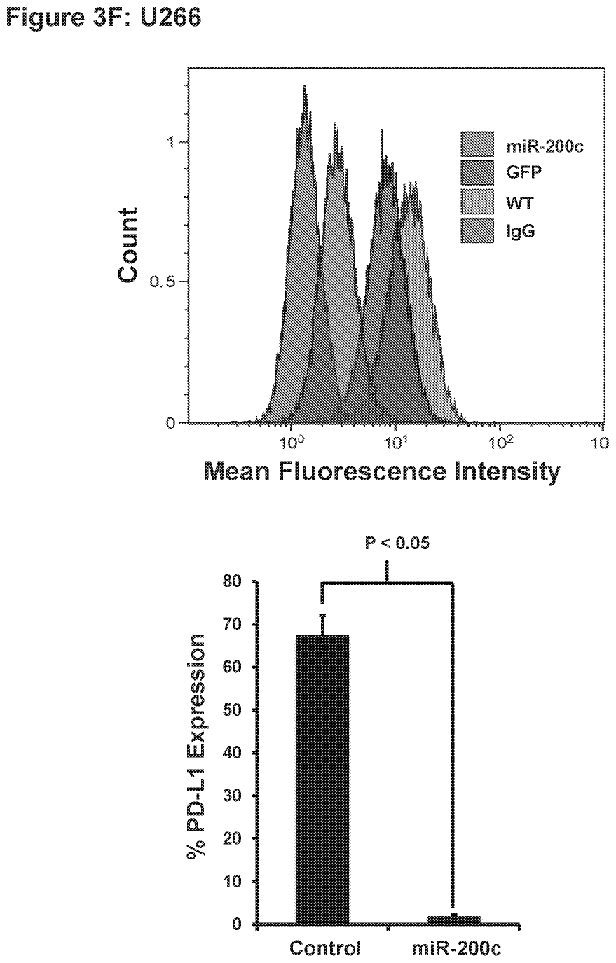

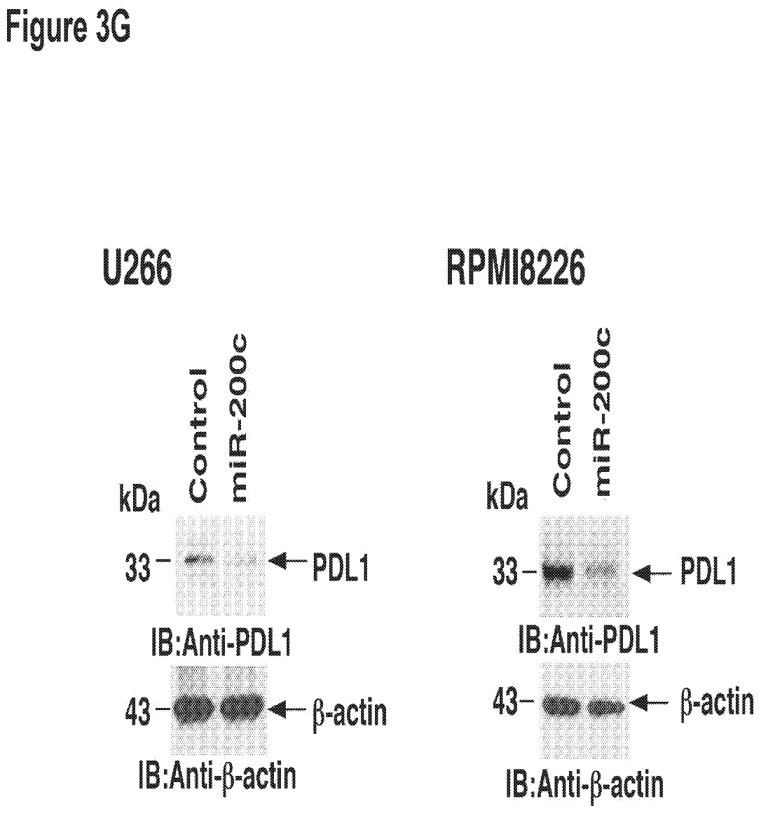

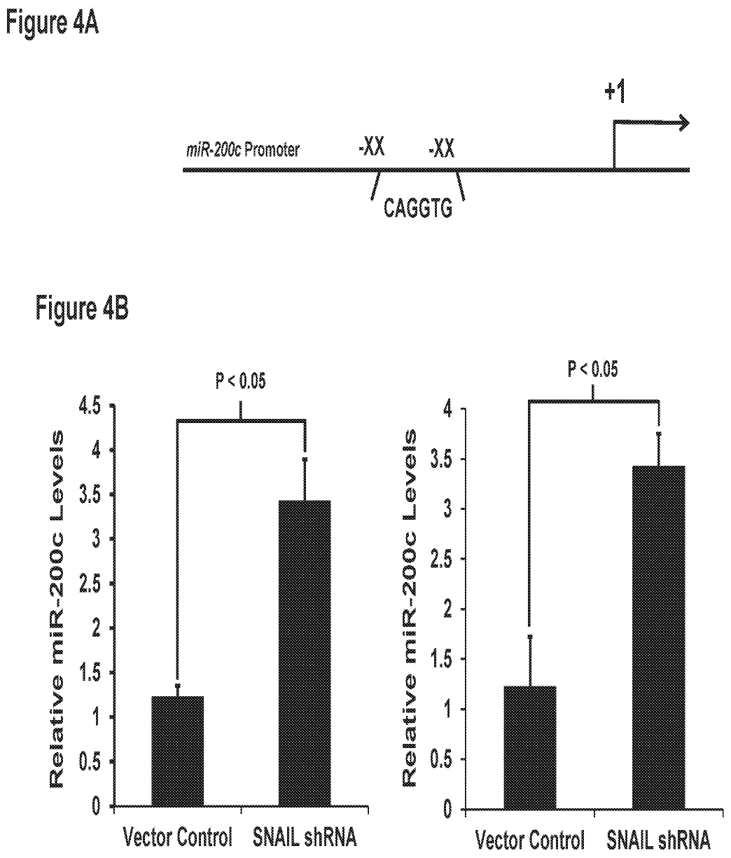

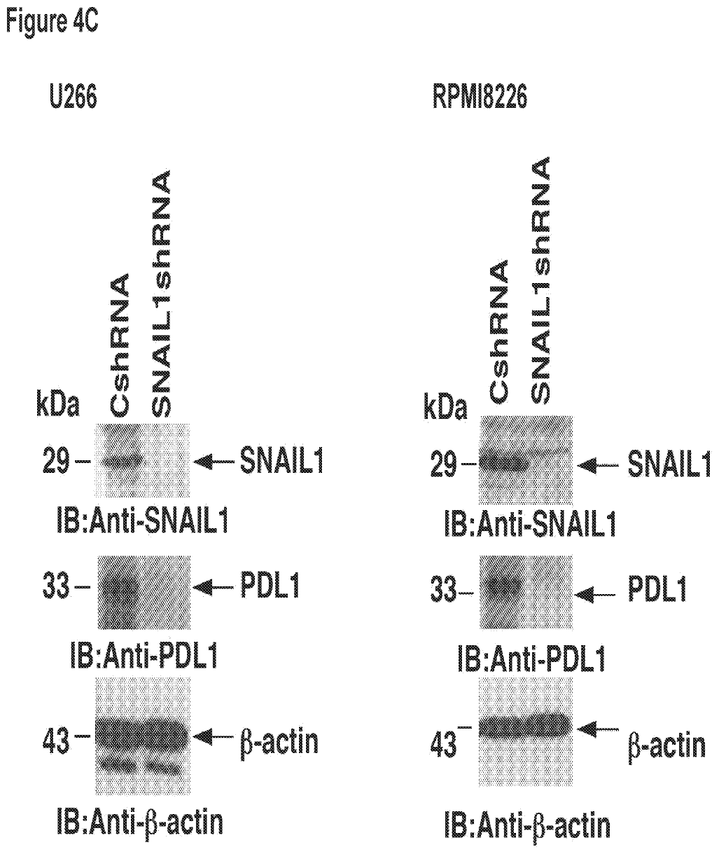

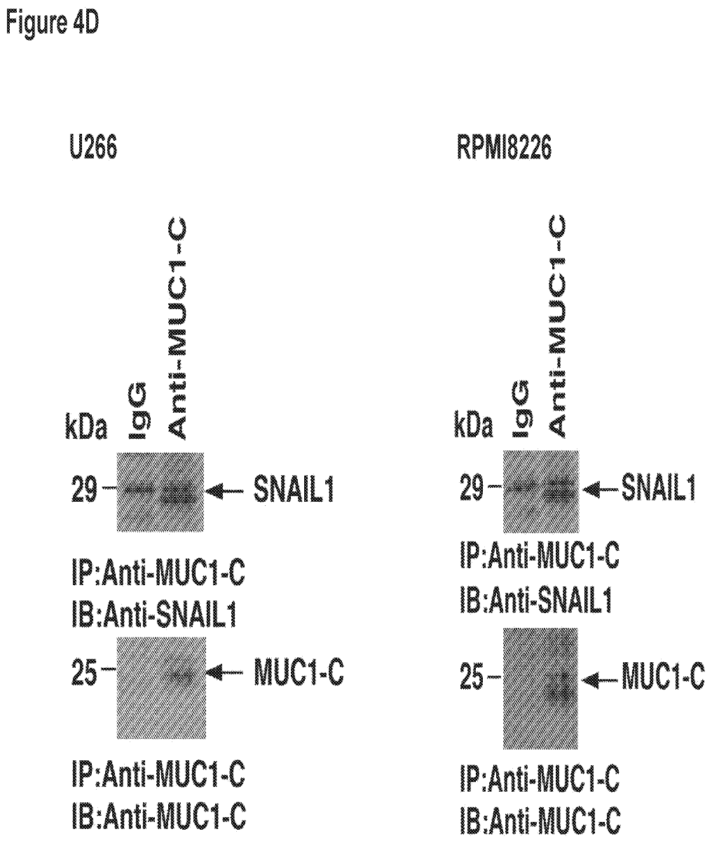

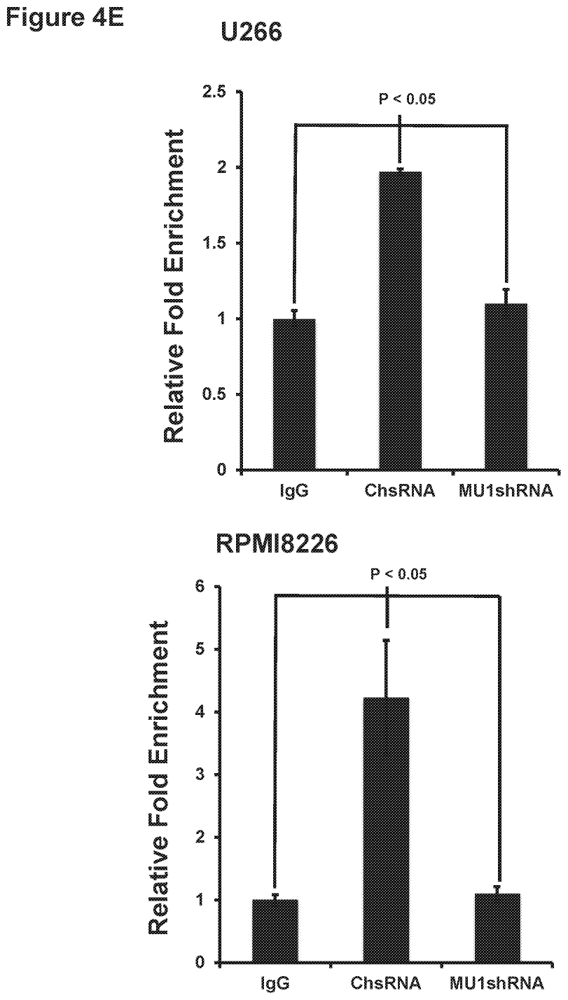

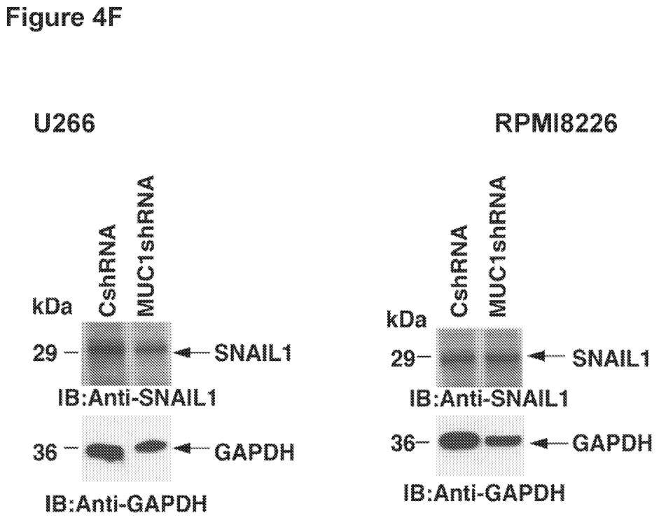

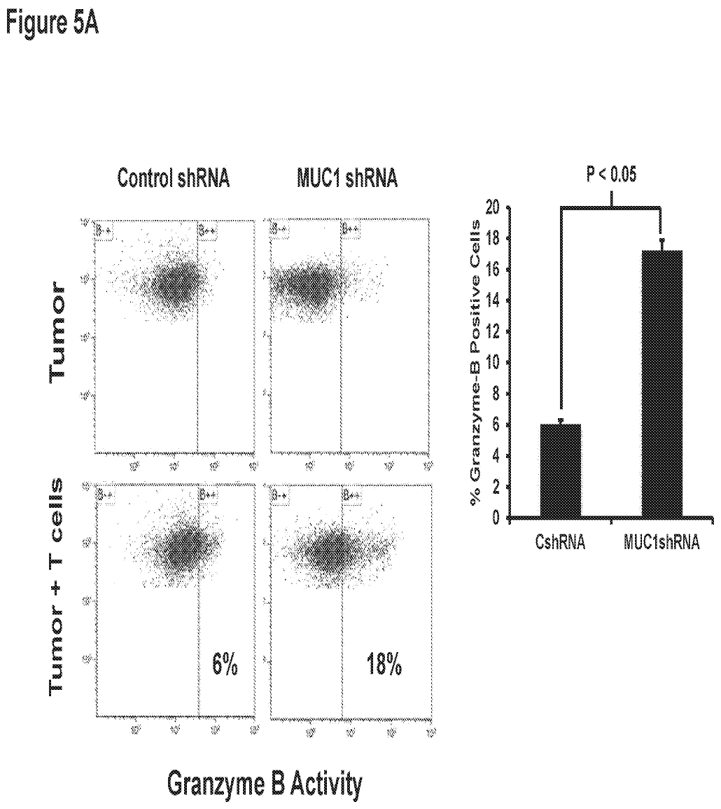

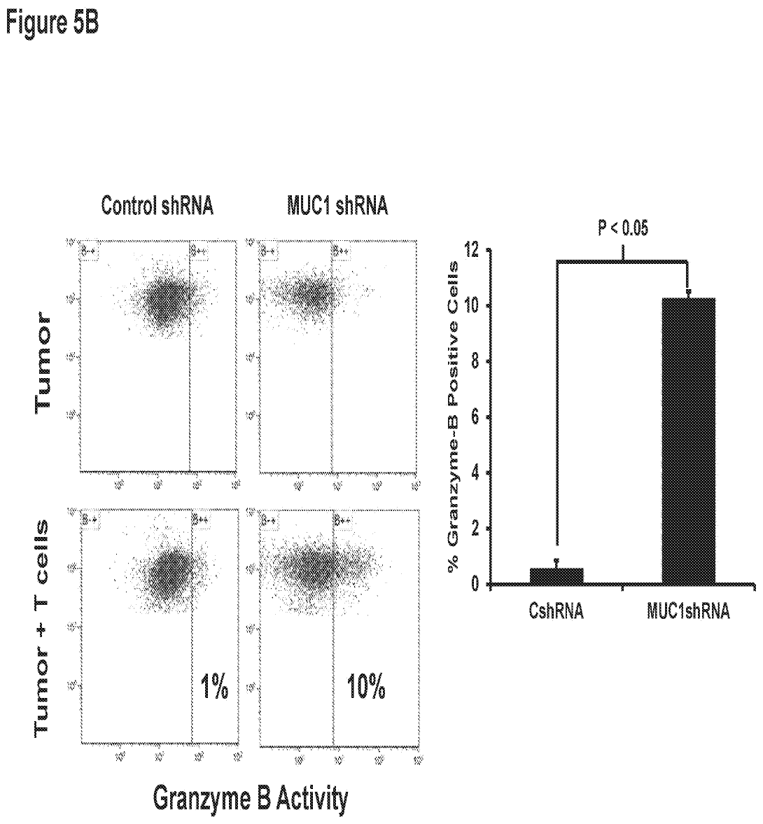

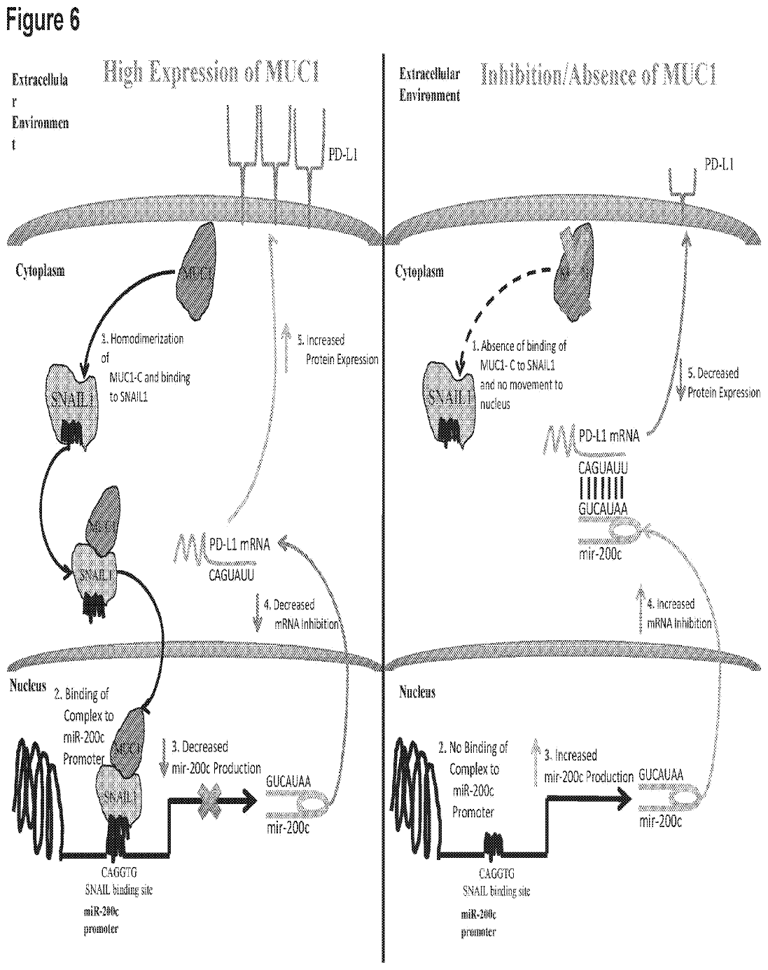

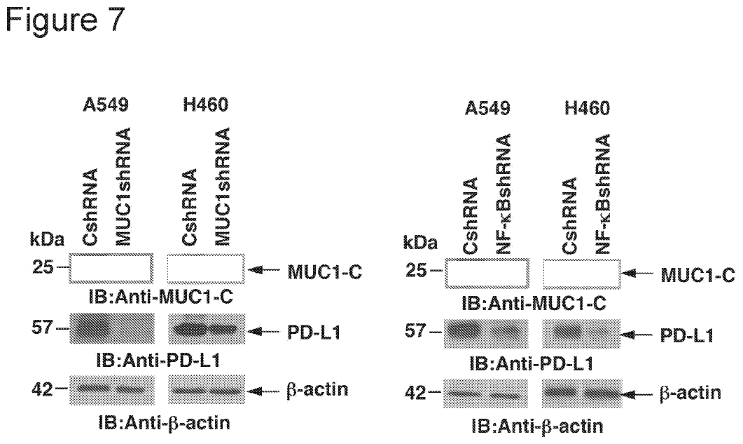

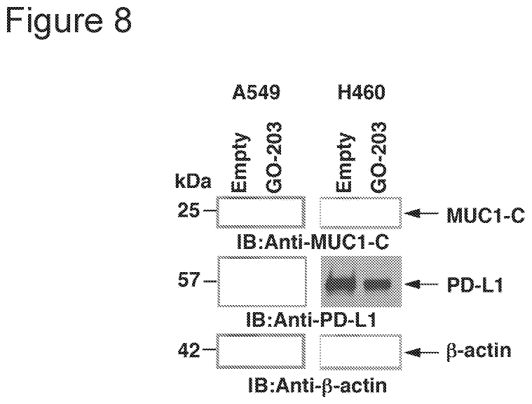

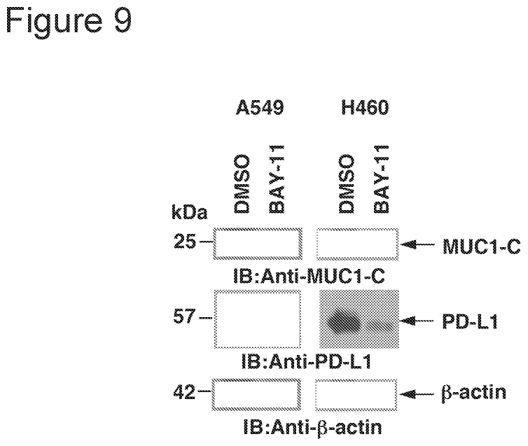

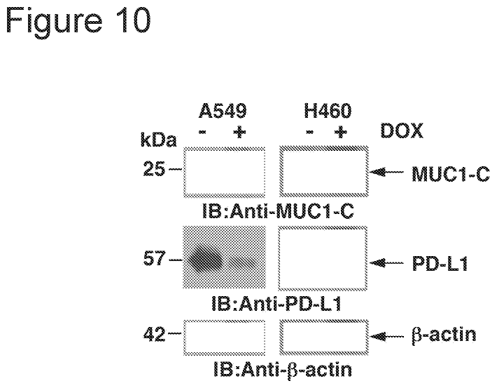

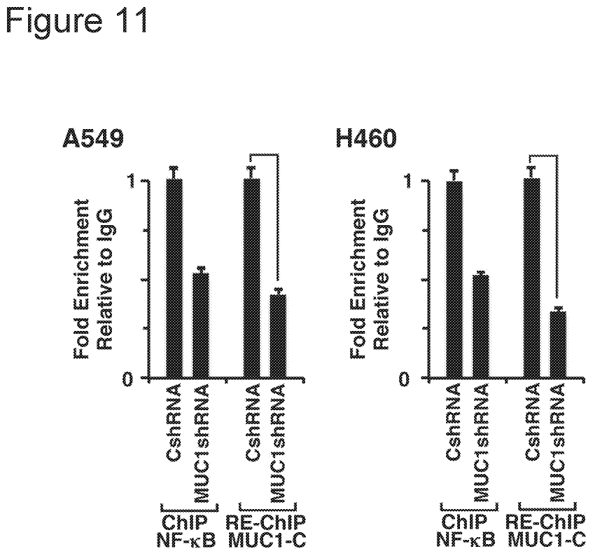

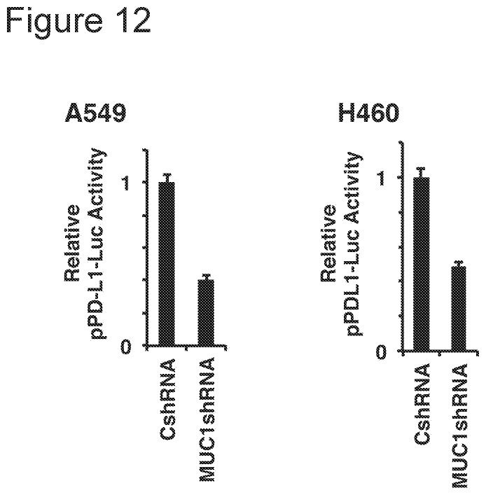

We next sought to elucidate the mechanism by which MUC1 regulates miR-200c expression. Previous studies in mammary epithelial cells demonstrated that the ZEB1 transcription suppressor binds to GC rich E-box elements (CACGTG) on the miR-200c promoter down-regulating expression of miR-200c (Rajabi et. al.). While ZEB1 was not identified in MM cells, an alternative epigenetic regulatory protein, SNAIL, has also been shown to bind the GC rich E-box elements in malignant cells (Ref. Can cell-FBP1). Consistent with its role in blocking transcription of miR-200c, we demonstrated that silencing of SNAIL via lentiviral transfection of shRNA resulted in the marked increase levels of miR-200c in RPMI cells (FIG. 3B, left). ChIP analysis of RPMI cells demonstrated occupancy of SNAIL on the miR-200c promoter in a MUC1 dependent manner (FIG. 3A left). Silencing of MUC1 via shRNA transfection resulted in the marked decrease in SNAIL binding to the miR200c promoter. Intriguingly the silencing of MUC1 has no effect on the expression of SNAIL protein in these cells (FIG. 3A right). Similar findings were observed in studies of the U266 human myeloma cell line. These findings are consistent with the role of MUC1 in stabilizing the interaction of SNAIL and the miR-200c promoter rather than a direct effect of SNAIL production.

### Example 5: MUC1 Oncoprotein Regulates PD-L1 Expression In AML Cells

We have demonstrated that MUC1 is a critical regulator of PDL1 expression on AML cells. Silencing of MUC1 expression was documented following lentiviral transfection with MUC1 specific shRNA or CRISPR mediated disruption of MUC1 translation. MUC1 silencing results in the near abrogation of MUC1 expression by the human AML cell lines, MOLM-14 and THP1 as determined by flow cytometric and western blot analysis. Silencing of MUC1-C on MOLM14 and THP1 AML cells resulted in a 2-fold increase in susceptibility to T cell mediated lysis as determined by a flourochrome based CTL assay.

### Example 6: Effect Of MUC1-C Silencing On PD-L1 Expression In-Vivo

To assess the effect of MUC1-C silencing on PD-L1 expression in-vivo, C57BL/6J mice were challenged with 100,000 GFP transfected TIB-49 murine AML cells in which MUC1-C was silenced using a lentiviral shRNA hairpin against MUC1-C. Following leukemia establishment, TIB-49 GFP+ cells were isolated from the bone marrows and spleens and PD-L1 expression on leukemic cells was measured. TIB-49 GFP+ cells of mice engrafted with MUC1 silenced AML cells demonstrated significantly lower PD-L1 expression compared to mice inoculated with TIB-49 cells transduced with a control vector (18% versus 3%; n=4). T cells isolated from bone marrow of mice inoculated with AML with silenced MUC1-C demonstrated a threefold increase in INF-γ production when stimulated ex-vivo with autologous tumor lysate as compared to T cells from mice inoculated with control AML cells (n=4). Our group has developed a peptide inhibitor drug (G0-203), a cell-penetrating peptide that binds to the MUC1-C CQC motif, disrupting MUC1-C interaction with downstream effectors. As was observed following MUC1 silencing, bone marrow derived CD4+ and CD8+ T cells isolated from mice treated with G0203 were shown to have two fold increased intracellular IFN-γ production compared to control mice following ex vivo exposure to AML lysate (n=4).

### Example 7: PD-L1 3′UTR Has A miR-200c Binding Site And Respond To miR-200c Over Expression In AML

To investigate the mechanism by which MUC1 regulates PDL1 expression we examined the role of noncoding RNAs that have been shown to be effectors of oncogenic modulation. MicroRNAs (miRNAs) are a conserved class of small RNAs that post-transcriptionally regulate gene expression by interacting with the 3′ untranslated region (3′ UTR) of target mRNAs. The 3′UTR of the PD-L1 gene contains putative binding sites for miR-200 family of micro-RNAs, supporting the hypothesis that miR-200 plays a role in regulating the expression of PDL1.

In the present study, we evaluated the effect of interfering with MUC1-C mediated signaling on mir200c levels, expression of PD-L1, and susceptibility of leukemic blasts to immune mediated targeting. MUC1-C silenced MOLM-14 cells demonstrated a 2 fold increase in miR-200c expression, as demonstrated by qPCR and was associated with a reduction of PD-L1 expression from 77% to 13%. These data was confirmed in THP1 cells with PDL-1 expression decreasing from 95% to 40% following MUC1-C silencing. In support of the observation that miR200C is involved in the regulation of PD-L1 expression, lentiviral overexpression of miR200c in MOLM14 cells led to a decrease in PD-L1 expression from 90% to 2% as demonstrated by flow cytometric analysis.

### Other Embodiments

While the invention has been described in conjunction with the detailed description thereof, the foregoing description is intended to illustrate and not limit the scope of the invention, which is defined by the scope of the appended claims. Other aspects, advantages, and modifications are within the scope of the following claims.

We claim:

1. A method of treating a tumor in a patient comprising administering to said patient a composition comprising a MUC1 inhibitor in an amount sufficient to decrease tumor PD-L1 expression.

2. The method of claim 1, further comprising administering a checkpoint inhibitor.

3. The method claim 1 or 2, wherein said patient has received a population of autologous dendritic cell/tumor cell fusions (DC/tumor fusions).

4. The method of any one of the preceding claims, wherein the MUC1 inhibitor is GO-203.

5. The method of any one of the preceding claims, wherein the tumor is a solid tumor or a hematologic tumor.

6. The method of claim 5, wherein said solid tumor is a lung tumor, a breast tumor, or a renal tumor.

7. The method of claim 5, wherein the hematologic tumor is acute myeloid leukemia (AML) or multiple myeloma (MM).

8. The method of claim 1, wherein the checkpoint inhibitor is an A2AR, B7-H3/CD276, B7-H4/VTCN1, BTLA, CD27, CD28, CD40, CD122, CD137, CTLA-4, GITR, ICOS, IDO, KIR, LAG3, OX40, PD1, PD-L1, PD-L2, TIM-3, or VISTA inhibitor.

9. The method of claim 6, wherein the checkpoint inhibitor is an A2AR, B7-H3/CD276, B7-H4/VTCN1, BTLA, CD27, CD28, CD40, CD122, CD137, CTLA-4, GITR, ICOS, IDO, KIR, LAG3, OX40, PD1, PD-L1, PD-L2, TIM-3, or VISTA antibody.

10. The method of any one of the preceding claims, wherein the further comprising administering to said patient an agent that targets regulatory T cells

11. The method of any one of the preceding claims, further comprising administering to said patient an immunomodulatory agent.

12. The method of claim 11 where the immunomodulatory agent is lenalidomide, pomalinomide or apremilast.

13. The method of any one of the preceding claims, further comprising administering said patient a TLR agonist, CPG ODN, polyIC, or tetanus toxoid
